(19) United States
(12) Patent Application Publication
(10) Pub. No.: US 2026/0097000 A1
(43) Pub. Date: Apr. 9, 2026

(54) INTRANASAL DELIVERY OF CANNABINOIDS

(71) Applicant: NANOMERICS LIMITED, St Albans, Hertfordshire (GB)

(72) Inventors: Ijeoma UCHEGBU, St Albans, Hertfordshire (GB); Ilona KUBAJEWSKA, St Albans, Hertfordshire (GB); Andreas SCHATZLEIN, St Albans, Hertfordshire (GB)

(21) Appl. No.: 19/111,969

(22) PCT Filed: Sep. 15, 2023
(86) PCT No.: PCT/GB2023/052397

§ 371 Date: Mar. 14, 2025

(30) Foreign Application Priority Data
Sep. 16, 2022 (GB) 2213668.3

(51) Int. Cl. A61K 9/51; A61K 9/00; A61K 9/107; A61K 9/16; A61K 31/00

(52) U.S. Cl. CPC A61K 9/5161; A61K 9/0043; A61K 9/1075; A61K 9/1652; A61K 31/658

(57) ABSTRACT

The present invention relates to a method of treatment wherein a composition comprising a cannabinoid and an amphiphilic carbohydrate compound such as GCPQ is administered intranasally. The method of treatment is particularly suitable for use in central nervous system disorders. The invention further relates to the composition when formulated with other components in pharmaceutical compositions for use in therapy.

### FIELD OF THE INVENTION

The present invention relates to compositions and methods of treatment wherein a composition comprising an amphiphilic carbohydrate compound and a cannabinoid is intranasally delivered to the human or animal body.

### BACKGROUND OF THE INVENTION

Phytocannabinoids found in the Cannabis sativa plant have been known to display therapeutic potential. Many of these compounds can bind to a wide variety of biological targets of the endocannabinoid system in the body. The endocannabinoid system regulates cognition, pain sensation, appetite, memory, sleep, immune function and mood. These effects are largely mediated through two members of the G-protein coupled receptor family, cannabinoid receptors 1 and 2 (CB1 and CB2), out of which CB1 receptors regulate the central and peripheral nervous systems, as discussed in Millar, S. A. et al. (“Towards Better Delivery of Cannabidiol (CBD)”, Pharmaceuticals, 2020). In particular, cannabidiol (CBD), one of the main active phytocannabinoids, has shown potential as it has a good safety profile and does not induce psychotropic effects which can lead to substance abuse. CBD has demonstrated promise as an analgesic, anticonvulsant, muscle relaxant, anxiolytic, antipsychotic and has shown neuroprotective, anti-inflammatory, and antioxidant activity, among other currently investigated uses. Beneficial therapeutic effects have been reported in patients with inflammatory, neurodegenerative, and autoimmune diseases as well as in those with epilepsy and cancer. Millar et al showed that phase 3 clinical trials of the Epidiolex CBD product demonstrated a clinically significant improvement in refractory epilepsy due to Lennox-Gastaut syndrome and Dravet syndrome, which are among the two most difficult types of epilepsy to treat. In 2018, Epidiolex was approved by the FDA to be the first CBD-based product available on the US market for the treatment of those two rare forms of epilepsy.

However, CBD's therapeutic potential and development as an effective drug by the pharmaceutical industry is hindered by intrinsic characteristics such as low bioavailability and low water solubility. Cannabinoids, generally, have a very low solubility in water (only 0.7 μg/mL for CBD specifically) and are highly lipophilic. Consequently, CBD cannot be easily absorbed orally and, therefore, a large quantity is required to have a medicinal effect. Due to the highly lipophilic nature of CBD (Log P 6.3), it is most commonly supplied as an oil or alcoholic formulation. Oil suspensions designed for the oral and oromucosal routes of administration have been approved for use, such as Epidiolex® delivered orally in an oil solution, and Sativex as an oromucosal spray. However, the limitation of oral CBD delivery is first pass metabolism. One study showed that the 7-hydroxy-cannabidiol (7-OH-CBD) metabolite accounted for 40% of orally delivered CBD. Therefore, first pass metabolism represents a significant barrier in increasing the bioavailability of CBD following oral administration (Millar et a).

In recent years, nasal administration as a potential brain delivery route, enabling the bypassing of the BBB has been explored (Wang et al “I.F. Nose-to-Brain Delivery.”, J Pharmacol Exp Ther, 370:593-601, 2019). The intranasal administration of drugs has the potential to prevent peripheral degradation, eliminate the drawbacks associated with oral administration, and allow targeted delivery to the site of action (the brain) while reducing plasma exposure, thus eliminating peripheral side effects.

There is a need for compositions and methods of delivery of cannabinoids to humans other than by oral administration. The present invention provides for cannabinoid compositions for nose-to-brain delivery of the cannabinoid.

### SUMMARY OF THE INVENTION

In accordance with a first aspect of the invention there is provided a method of treatment wherein a composition comprising an amphiphilic carbohydrate compound and a cannabinoid is intranasally delivered to the human or animal body.

More particularly the invention provides a method of treating a disease of the central nervous system in a human comprising administering to the human a composition comprising a cannabinoid and an amphiphilic carbohydrate compound, wherein the composition is intranasally administered to a human or animal body.

In accordance with a second aspect of the invention, there is provided a pharmaceutical composition suitable for intranasal administration comprising an amphiphilic carbohydrate compound and a cannabinoid and one or more pharmaceutically acceptable excipients.

In this invention, where methods of treatment are mentioned, it is also intended that the invention covers compositions comprising an amphiphilic carbohydrate compound and a cannabinoid for use in those methods or in therapy. For instance the invention therefore provides a composition comprising an amphiphilic carbohydrate compound and a cannabinoid for use in a method of treatment wherein the composition is intranasally delivered to the human or animal body.

More particularly the invention provides a composition comprising a cannabinoid and an amphiphilic carbohydrate compound for use in a method of treating a disease of the central nervous system in a human by intranasal administration. Typically the disease is epilepsy. In alternative embodiments the treatment may be of pain, anxiety and/or autoimmune diseases.

Also provided is the use of the above compositions in the manufacture of a medicament for use in therapy.

The method of treatment according to the invention resolves some of the issues experienced in the art by enabling a greater concentration of the therapeutic cannabinoid to be delivered to the brain than conventional oral administration methods, while reducing dose drainage to the lungs or stomach. The treatment involves administering a therapeutically effective amount of the cannabinoid to the brain via the nose. The formulations can effectively treat a range of central nervous system diseases including epilepsy and pain disorders. Further advantageously, the intranasal method of administration according to the invention also incurs fewer drawbacks, such as peripheral degradation. The composition can be formulated so that it is conveniently compatible with nasal sprays, facilitating easy and effective intranasal delivery directly to the brain via the olfactory nerve. The main side effects of oral cannabidiol include diarrhoea, vomiting and an increase in liver enzymes, and these side effects may be alleviated by using a delivery method that avoids the gastrointestinal tract.

### BRIEF DESCRIPTION OF THE FIGURES

FIG. 1: Schematic workflow of preparing a GCPQ-CBD formulation in water.

FIG. 2: Calibration curve (3-100 μg/mL) and peak overlay showing linearity and quantification range of CBD (A) and representative integration in MET-CBD formulation sample showing detection at retention time of ˜4 min (B). Overlaid chromatograms of peaks obtained from CBD solutions of ascending concentration as depicted in the inset calibration curve graph.

FIG. 3: Colloidal properties of CBD-GCPQ formulations prepared at 1:10 g g-1 (A) and 1:5 g g-1 (B) CBD to GCPQ ratios, resuspended in water at concentrations of 1 mg/mL or 5 mg/mL (of CBD equivalent), showing size distribution by intensity and size (Z-average mean), polydispersity (PDI) and zeta potential (ZP) parameters. Data are expressed as mean±SEM of 3 measurements. Each line corresponds to a different batch of the formulation.

FIG. 4: Colloidal parameters of freshly prepared CBD-GCPQ (1:5 g g-1) formulations (n=3) in water at concentration of ˜5 mg/mL (of CBD equivalent) prior to spray-drying (“pre-SD”) and compared with rehydrating the powder at the same ˜5 mg/mL concentration, showing size distribution by intensity, size (Z-average) and polydispersity (PDI) parameters. Data are expressed as mean±SEM of 3 replicate batches (3 measurements each). Each line corresponds to a different batch of the formulation.

FIG. 5A: A graph showing the size distribution D₅₀ of the spray-dried CBD-GCPQ microparticles at 0, 7, 14 and 30 days under RT and 4° C. storage.

FIG. 5B: A graph showing the % of the spray-dried CBD-GCPQ particles below 10 μm at 0, 7, 14 and 30 days under RT and 4° C. storage.

FIG. 6A: A graph showing the average zeta potential values of the spray-dried CBD-GCPQ particles on reconstitution in water at 0, 7, 14 and 30 days under RT and 4° C. storage.

FIG. 6E: A graph showing the polydispersity of the spray-dried CBD-GCPQ particles on reconstitution in water at 0, 7, 14 and 30 days under RT and 4° C. storage.

FIG. 6C: A graph showing the CBD concentration in mg/mL of the spray-dried CBD-GCPQ particles on reconstitution in water at 0, 7, 14 and 30 days under RT and 4° C. storage.

FIG. 7: Calibration curve (1-500 ng/mL) and peak overlay showing linearity and quantification range of spiked CBD in mobile phase validating the MRM method in non-biological sample (A) and representative integration showing area under peak (AUP) and signal-to-noise ratio (SNR) of lowest concentration 1 ng/mL (B). Overlaid chromatograms of peaks obtained from CBD solutions of ascending concentration as depicted in the inset calibration curve graph.

FIG. 8A: A calibration curve of AUC ratio v Concentration of CBD in the brain (1-100 ng/mL).

FIG. 8E: A calibration curve of AUC ratio v Concentration of CBD in the plasma (3-200 ng/mL).

FIG. 9: Colloidal stability and pH measurements of CBD-GCPQ (1:5) formulations prepared for in vivo dosing, rehydrated at a concentration of 5 mg/mL (of CBD equivalent), showing size distribution by intensity, size (Z-average mean) and polydispersity (PDI) parameters at initial pH (=4.5-4.8) and after pH adjustment with NaOH to pH=5.5-5.6. Each curve represents a separate batch (n=3).

FIG. 10A: A graph of CBD concentration v Time of the in vivo pharmacokinetic studies in rats showing CBD levels over 2 hours in the brain (top left).

FIG. 10B: A graph of CBD concentration v Time of the in vivo pharmacokinetic studies in rats showing CBD levels over 2 hours in the olfactory bulbs (top right).

FIG. 10C: A graph of CBD concentration v Time of the in vivo pharmacokinetic studies in rats showing CBD levels over 2 hours in the plasma (bottom).

### DETAILED DESCRIPTION OF INVENTION

The present invention relates to compositions and methods of treatment wherein a composition comprising an amphiphilic carbohydrate compound and a cannabinoid is intranasally delivered to the human or animal body.

The amphiphilic carbohydrate compounds in the compositions and methods of this invention may form particulate aggregates. These may be formed by the aggregation of individual amphiphile molecules and have a mean particle size of between 10 nm and 50 μm. The mean particle size can readily be determined microscopically or by using photon correlation spectroscopy and is conveniently determined in aqueous dispersions prior to filtration. The polymeric micellar aggregates may have a minimum mean particle size of at least 10 nm, and more preferably at least 30 nm, and a maximum mean particle size which is preferably 10 μm or less. The mean particle size may be between 10 nm to 50 μm or between 10 nm and 20 μm or between 10 nm and 5 μm or between 10 nm to 1 μm or between 10 nm and 500 nm or between 10 nm and 100 nm or between 20 nm to 50 μm or between 20 nm and 20 μm or between 20 nm and 5 μm or between 20 nm to 1 μm or between 20 nm and 500 nm or between nm and 100 nm or between 50 nm to 50 μm or between 50 nm and 20 μm or between 50 nm and 5 μm or between 50 nm to 1 μm or between 50 nm and 500 nm or between 10 nm and 100 nm.

In the invention, the amphiphilic carbohydrate compound is formulated with a cannabinoid drug. The amphiphilic carbohydrate compound is capable of self-assembly into nanoparticles. Pharmaceutical compositions of the present invention may comprise nanodispersions of the nanoparticles of the amphiphilic carbohydrate compound and drug.

The composition used in the method according to the invention is preferably in the form of nanoparticles, which may be further processed by different methods, including spray-drying (as further explained below) or by adding to powders to make granules or by lyophilisation, to form a nano-in-microparticle composition. The nano-in-microparticle composition may be in the form of a dry powder and is preferably colloidally stable on reconstitution in aqueous media. Preferably, the nano-in-microparticles have a spherical morphology. Nano-in-microparticles may also have a hollow morphology or may be oval or irregular in shape. Preferably, the mean size of the nanoparticles in the nano-in-microparticles composition is under 1000 nm, even more preferably wherein the mean size of the nanoparticles is under 500 nm. The mean particle size may be between 10 nm to 1 μm or between 10 nm and 750 m or between 10 nm and 500 nm or between 10 nm to 250 nm or between 20 nm to 1 μm or between 20 nm and 750 nm or between 20 nm and 500 nm or between 20 nm to 250 nm or between 50 nm to 1 μm or between 50 nm and 750 nm or between 50 nm and 500 nm or between 50 nm to 250 nm.

The size distribution of the microparticles in the nano-in-microparticulate composition may be measured according to D₁₀, D₅₀ and D₉₀. D₁₀ represents the point on the distribution curve below which 10% of the particles fall; D₅₀ is the median particle size (or volume) distribution and represents the point on the distribution curve below which 50% of the particles fall; and D₉₀ represents the point on the distribution curve below which 90% of the particles fall. The size distribution according to these parameters can also be calculated by laser scattering and/or microscopic methods such as scanning electron microscopy.

The median volume distribution, D₅₀, of the microparticles according to the present invention is preferably between 5 to 30 μm, more preferably between 10 to 25 μm. The D₁₀ size distribution of the microparticles may be below 15 μm. The D₁₀ size distribution is preferably above 10 μm to avoid lung deposition.

The median volume distribution, D₅₀, of the microparticles in the nano-in-microparticulate composition is typically between 5 to 30 μm, preferably between 10 to 25 μm. Generally, less than 10% of particles are below 10 μm. In a further preferred embodiment, the polydispersity of the nanoparticles that make up the nano-in-microparticles is less than 0.5, preferably less than 0.2, and more preferably less than 0.1. The polydispersity may be between 0.01 to 0.5 or 0.01 to 0.3 or 0.01 to 0.1 or from 0.05 to 0.5 or 0.05 to 0.3 or 0.05 to 0.1 or from 0.1 to 0.3 or 0.1 to 0.2.

The amphiphilic carbohydrate compound of the composition is preferably a chitosan derivative, more preferably comprising units represented by the general formula (I):

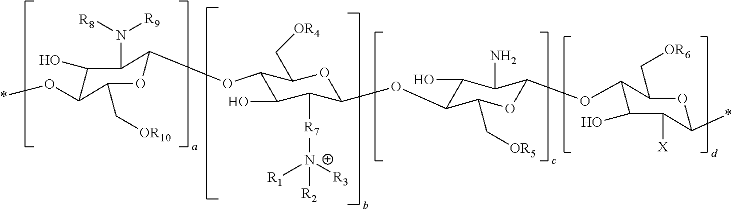

wherein a+b+c+d=1.000 and
a is between 0.01 and 0.970
b is between 0.01 and 0.990,
c is between 0.001 and 0.970, and
d is between 0.01 and 0.990;
and wherein:
X is a hydrophobic group;
R₁, R₂ and R₃ are independently selected from a substituted or unsubstituted alkyl group;
R₄, R₅, R₆ and R₁₀ are independently selected from hydrogen, a substituted or unsubstituted alkyl group, a substituted or unsubstituted ether group, or a substituted or unsubstituted alkene group;
R₇ may be present or absent and, when present, is an unsubstituted or substituted alkyl group, an unsubstituted or substituted amine group or a substituted or unsubstituted amide group;
R₃ and R₉ are independently selected from hydrogen and either a substituted or unsubstituted alkyl group, a substituted or unsubstituted ether group, or a substituted or unsubstituted alkene group;
or a salt thereof.

In an alternative embodiment in the general formula I above,
a is between 0.00 and 0.970
b is between 0.01 and 0.990,
c is between 0.000 and 0.970, and
d is between 0.01 and 0.990; wherein a+b+c+d=1.000.

In the above general formula, the a, b, c and d units may be arranged in any order and may be ordered, partially ordered or random. There may be more than one type of each a, b, c and d unit comprising different R groups. The * in the formula is used to indicate the continuing polymer chain.

In preferred embodiments, the molar proportion of the d units is greater than 0.01, and more preferably is at least 0.110, more preferably is at least 0.120, more preferably is at least 0.150 or in some embodiments is at least 0.18. Generally, the molar proportion of the d unit is 0.500 or less, and more preferably is 0.350 or less.

Preferably, the molar proportion of the b unit is between 0.010 and 0.800, and more preferably between 0.050 and 0.600.

Preferably, the molar proportion of the c unit is between 0.0200 and 0.850, and more preferably between 0.05 and 0.550.

Preferably the molar proportion of the a unit is between 0.05 and 0.85 and more preferably between 0.10 and 0.75.

The d units provide the first portion of the monomer units that are derivatised with a hydrophobic group, and the b units provide the second portion of the monomer units and are derivatised with a quaternary nitrogen group. The a units provide the third group of monomer units in which the amine groups are derivatised in a different manner to the first or second group. There may be more than one type of “a” group present (for instance there may be “a” groups differing in the R⁸ and R⁹ groups attached to the N atom).

The c units provide the fourth group of monomer units in which the amine groups are underivatised.

In the present invention, the hydrophobic group X is preferably selected from a substituted or unsubstituted group which is an alkyl group such as a C₄₋₃₀ alkyl group, an alkenyl group such as a C₄₋₃₀ alkenyl group, an alkynyl group such as a C₄₋₃₀ alkynyl group, an aryl group such as a C₅₋₂₀ aryl group, a multicyclic hydrophobic group with more than one C₄-C₈ ring structure such as a sterol (e.g. cholesterol), a multicyclic hydrophobic group with more than one C₄-C₈ heteroatom ring structure, a polyoxa C₁-C₄ alkylene group such as polyoxa butylene polymer, or a hydrophobic polymeric substituent such as a poly (lactic acid) group, a poly(lactide-co-glycolide) group or a poly(glycolic acid) group. The X groups may be linear, branched or cyclo groups. Any of the X groups may be directly linked to the d unit (i.e. at the C2 of the monomer unit), or via a functional group such as an amine group, an acyl group, or an amide group, thereby forming linkages that may be represented as X′-ring, X′—NH—, X′—CO-ring, X′CONH-ring, where X′ is the hydrophobic group as defined above.

Preferred examples of X groups include those represented by the formulae CH₃(CH₂)ₙ—CO—NH— or CH₃(CH₂)ₙ—NH— or the alkeneoic acid CH₃ (CH₂)ₚ—CH═CH—(CH₂)_{q}—CO—NH—, where n is between 4 and 30, and more preferably between 6 and 20, and p and q may be the same or different and are between 4 and 16, and more preferably 4 and 14. A particularly preferred class of X substituents are linked to the chitosan monomer unit via an amide group, for example as represented by the formula CH₃(CH₂)ₙCO—NH—, where n is between 2 and 28. Examples of amide groups are produced by the coupling of carboxylic acids to the amine group of chitosan. Preferred examples are fatty acid derivatives CH₃(CH₂)ₙCOOH such as those based on capric acid (n=8), lauric acid (n=10), myristic acid (n=12), palmitic acid (n=14), stearic acid (n=16) or arachidic acid (n=18).

In the above formula, R₁, R₂ and R₃ are preferably independently selected from a substituted or unsubstituted alkyl group such as a C₁₋₁₀ alkyl group. R₁, R₂ and/or R₃ may be linear or branched. Preferably, R₁, R₂ and R₃ are independently selected from methyl, ethyl or propyl groups.

In the above formula, R₃ and R₉ are preferably independently selected from hydrogen and a substituted or unsubstituted alkyl group such as a C₁₋₁₀ alkyl group. R₃ and/or R₉ may be linear or branched. Preferably, R₃ and R₉ are independently selected from methyl, ethyl or propyl groups.

In the above formula, R₄, R₅, R₆ and R₁₀ present on the C6 or the sugar units are independently selected from hydrogen, a substituted or unsubstituted alkyl group, a substituted or unsubstituted ether group, or a substituted or unsubstituted alkene group. Preferred R₄, R₅, R₆ and R₁₀ groups are substituted with one of more hydroxyl groups, or another non-ionic hydrophilic substituent. Examples of R₄, R₅, R₆, and R₁₀ groups are represented by the formulae —(CH₂)ₚ—OH, where p is between 1 and 10, and is preferably between 2 and 4, or —(CH₂)ₚ—CH(CH₂—OH)₂ where p is between 1 and 10 or —(CH₂)ₚ—C(CH₂—OH)ᵣ where p is between 1 and 10, and r is 3, or —(CH₂CH₂OH)ₚ, where p is between 1 and 300.

The R₇ group may be present or absent in the general formula. When absent, it provides a quaternary ammonium functional group that is directly linked to the monomer unit of the chitosan backbone. When the R₇ group is present it may be a unsubstituted or substituted alkyl group (e.g. a C₁₋₁₀ alkyl group) for example as represented by the formula —(CH₂)ₙ—, an amine group as represented by the formula —NH—(CH₂)ₙ—, or an amide group as represented by the formula —NH—CO—(CH₂)ₙ—, where n is 1 to 10 and is preferably 1 to 4. A preferred example of the R₇N⁺R₁R₂R₃ substituent is provided by coupling betaine (—OOC—CH₂—N—(CH₃)₃) to the amine substituent of the b unit providing an amide group such as in: —NH—CO—CH₂—N⁺R₁R₂R₃.

As indicated, some of the substituents described herein may be either unsubstituted or substituted with one or more additional substituents as is well known to those skilled in the art. Examples of common substituents include halo; hydroxyl; ether (e.g., C₁₋₇ alkoxy); formyl; acyl (e.g. C₁₋₇ alkylacyl, C₅₋₂₀ arylacyl); acylhalide; carboxy; ester; acyloxy; amido; acylamido; thioamido; tetrazolyl; amino; nitro; nitroso; azido; cyano; isocyano; cyanato; isocyanato; thiocyano; isothiocyano; sulfhydryl; thioether (e.g., C₁₋₇ alkylthio); sulphonic acid; sulfonate; sulphone; sulfonyloxy; sulfinyloxy; sulfamino; sulfonamino; sulfinamino; sulfamyl; sulfonamido; C₁₋₇ alkyl (including, e.g., unsubstituted C₁₋₇ alkyl, C₁₋₇ haloalkyl, C₁₋₇ hydroxyalkyl, C₁₋₇ carboxyalkyl, C₁₋₇ aminoalkyl, C₅₋₂₀ aryl-C₁₋₇ alkyl); C₃₋₂₀ heterocyclyl; and C₅₋₂₀ aryl (including, e.g., C₅₋₂₀ carboaryl, C₅₋₂₀ heteroaryl, C₁₋₇ alkyl-C₅₋₂₀ aryl and C₅₋₂₀ haloaryl) groups.

The term “ring structure” as used herein, pertains to a closed ring of from 3 to 10 covalently linked atoms, yet more preferably 3 to 8 covalently linked atoms, yet more preferably 5 to 6 covalently linked atoms. A ring may be an alicyclic ring, or aromatic ring. The term “alicyclic ring,” as used herein, pertains to a ring which is not an aromatic ring.

The term “carbocyclic ring”, as used herein, pertains to a ring wherein all of the ring atoms are carbon atoms.

The term “carboaromatic ring”, as used herein, pertains to an aromatic ring wherein all of the ring atoms are carbon atoms.

The term “heterocyclic ring”, as used herein, pertains to a ring wherein at least one of the ring atoms is a multivalent ring heteroatom, for example, nitrogen, phosphorus, silicon, oxygen or sulphur, though more commonly nitrogen, oxygen, or sulphur. Preferably, the heterocyclic ring has from 1 to 4 heteroatoms.

The above rings may be part of a “multicyclic group”.

In one embodiment the amphiphilic carbohydrate compound may comprise an additional group, an acyl group A:

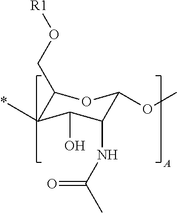

This group A may be present from 0.5% to 30 mole % and the amounts of the remaining units may be adjusted accordingly such that a has a range of 0.05 to 40 mole %, b has a range of 5 to 20 mole %, c has a range of 0.05 to 20 mole % and d has a range of 5 to 30 mole %.

A preferred amphiphilic carbohydrate compound of the invention has formula:

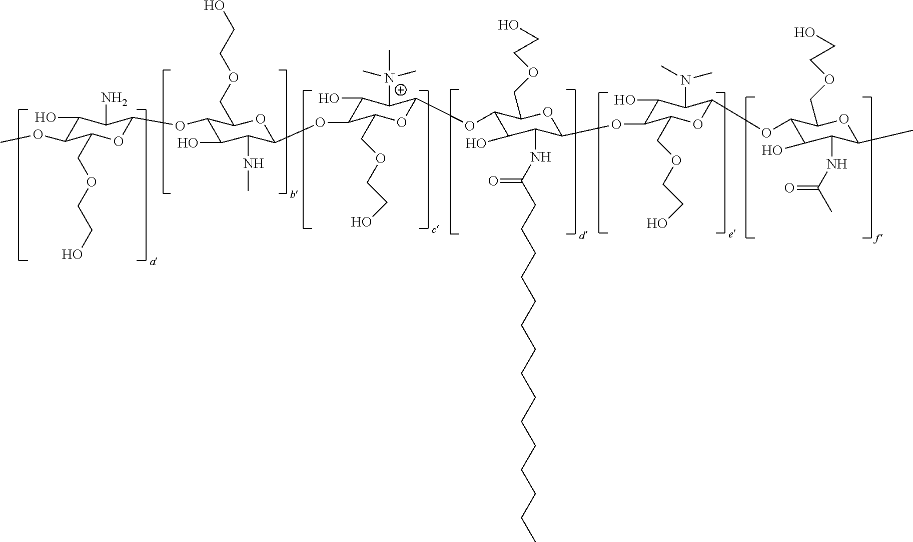

wherein:
unit a′ corresponds to unit c according to the formula I;
units b′ and e′ together correspond to unit a according to the formula I;
unit c′ corresponds to unit b according to the formula I;
unit d′ corresponds to unit d according to the formula I;
unit f corresponds to the unit A above;
the proportion of units a′+b′+c′+d′+e′+f′=1 and the preferred amounts of each unit are as outlined above;
or salt thereof.

Preferably, the amphiphilic carbohydrate compound is a quaternary ammonium palmitoyl glycol chitosan (GCPQ).

Preferably, the amphiphilic carbohydrate compound is N-palmitoyl-N-monomethyl-N,N-dimethyl-N,N,N-trimethyl-6-O-glycolchitosan, otherwise known as quaternary ammonium palmitoyl glycol chitosan (GCPQ).

In this case, the palmitoylation level (corresponding to group d) of the GCPQ is preferably between 5-50% per monomer, for instance, 10-25% per monomer. In one embodiment the d level is less than or equal to 31% per monomer, and is preferably in the range 11 to 31%, even more preferably in the range 11 to 20%.

The quaternisation level (b) is preferably between 3-40% per monomer, preferably 10-30% per monomer. In one embodiment the b level is less than or equal to 17% per monomer and is preferably in the range 8 to 17%.

Percentage levels where given are mole %.

The molecular weight of the amphiphilic carbohydrate compound, typically GCPQ may be in the range 1-40 kDa or 1-30 kDa, for instance 5-30 kDa or 10-30 kDa or 8-20 kDa. Even more preferably the molecular weight is around 10-15 kDa.

The method according to the invention can be used for treating infectious and autoimmune diseases of the central nervous system.

The cannabinoid is preferably a non-psychoactive cannabinoid. By non-psychoactive cannabinoid is meant a compound derived from the Cannabis sativa plant which does not induce changes in perception, behaviour or mental processes. For example, a non-limiting list of non-psychoactive cannabinoids suitable for use in the invention includes: cannabidiol (CBD), cannabigerol (CBG), cannabidiolic acid (CBDA), cannabidivarin (CBDV), and cannabichromene (CBC). Preferably, the non-psychoactive cannabinoid according to the invention is cannabidiol.

Cannabinoids may be used as an analgesic, anticonvulsant, muscle relaxant, anxiolytic or antipsychotic agent. They may also have neuroprotective, anti-inflammatory, and antioxidant activity. Accordingly the invention may be put to any of these uses.

The invention has particular utility for the delivery of non-psychoactive cannabinoids to the brain. Non-psychoactive cannabinoids, delivered in accordance with the invention, are able to have a therapeutic effect in the brain. Cannabinoids have been shown to be particularly useful against diseases of the central nervous system, particularly infectious and autoimmune diseases of the central nervous system (CNS).

The composition according to the invention may be used to treat a number of disorders, including epilepsy, Dravet's syndrome, Bell's palsy, cerebral palsy, Alzheimer's disease, dementia, motor neurone disease, multiple sclerosis, Parkinson's, long covid, neurofibromatosis, shingles, sciatica, pain and sleep disorders such as migraines, Lennox-Gastaut syndrome, psychiatric diseases, neurodegenerative conditions, and brain cancers.

Preferred conditions for treatment in accordance with the invention are epilepsy, pain, anxiety and autoimmune diseases.

The method of treatment according to the invention involves the intranasal delivery to the animal or human body of a composition comprising a cannabinoid and amphiphilic carbohydrate compound. Intranasal administration of drugs provides for enhanced targeted delivery to the brain, while eliminating unwanted side effects, which may be common with other forms of administration such as the oral route. Targeted delivery to the brain is achieved by bypassing the blood-brain-barrier (BBB) and delivering drugs to the top of the nose to be transported along the olfactory nerve.

By using an amphiphilic carbohydrate compound, a cannabinoid composition can be developed and suitably formulated into nanoparticles or preferably into nano-in-microparticles for compatibility with nasal drug delivery devices that administer compound nasally. Preferably the drug is cannabidiol and the amphiphilic carbohydrate compound is GCPQ. The composition may be suitable for nasal sprays. By using this particular approach, dose drainage to the lungs or stomach upon nasal administration is significantly reduced.

The device may work as described below.

The formulations of the present invention may dispensed from a dispenser of a fluid, in particular a gas borne solid or liquid particulate, having: a container for a fluid; a chamber for containing a particulate, in use in fluidic communication with the container; means to cause the fluid to move into the chamber from the container; means as necessary to cause the fluid to engage with particulate accommodated within the chamber to de-aggregate it if aggregated and to agitate it into turbulent flow to produce a mobile fluid comprising the particulate; a discharge outlet, capable of being placed in fluidic communication with the chamber; and a release means for release of the mobile fluid from the dispenser through the discharge outlet. When used herein the term ‘turbulent flow’ includes cyclonic or vortical flow, which are preferred forms of turbulent flow. The dispenser may be used and will operate in any orientation including upright, inverted or laid-down fashion. Another advantage is that it solves the problem of de-aggregating and/or fluidizing particulates using relatively uncomplicated and potentially inexpensive technology. The particulate is generally a solid particulate and is useful for dispensing particulate compositions comprising a medicament in metered doses, especially if the particulate is a solid particulate that tends to clump fairly readily in storage and/or in transit. The properties of any particulate may be any within a wide variety that are compatible with the function of the present device, such as its density, particle size, specific surface area, the desired dose, etc. Preferably the particles have a narrow size distribution and are of similar shape.

The fluid in the container may be any that is a sufficiently mobile fluid to agitate the particulate into turbulent flow, and is stable during storage, and is inert to the particulate and the dispensing target.

The fluid may be a gas, such as air, or if the particulate is not inert longer-term to air, nitrogen, a conventional optionally fluorinated lower hydrocarbon propellant, such as a hydroflurocarbon (HFC) or carbon dioxide; a liquid, such as water, or if the particulate is not inert longer-term to water, a (usually pressurized) conventional optionally fluorinated lower hydrocarbon propellant, such as butane or an HFC, a hydrofluoro alkane (HFA) propellant or any compatible combination thereof.

The fluidic communication between container and chamber often comprises at least one channel, and preferably at least a pair of channels, which runs between the container and the chamber. Any channels that run between the container and the chamber may take the form of conduits, ducts or tubes. The shape and size of any channels for agitating the particulate with the fluid may be any within a wide variety that are compatible with that function. These may be dependent on the particular properties of the particulate, such as its density, particle size, specific surface area, the desired dose, etc, and may be of circular cross section and/or any other regular curved cross-section, e.g. a generally elliptical, semicircular or semielliptical cross-section. However, often each will be of rectilinear cross-section, such as in the form of a slot or triangular, square or oblong cross-sectional duct. For a quantity of a composition comprising a medicament provided in the fully laden present dispenser of 0.5-35 mg, each channel will typically have a cross-sectional area of 0.03 to 3.0 mm² and in particular 1.0 to 1.5 mm². The channels may be of widely varying shape along their length, e.g. curved, but typically are straight.

Where there is at least a pair of channels, all the channels will typically have similar and often identical dimensions and configurations. Channels in a pair of channels may of course have opposite handedness where appropriate.

The container is a pressurized fluid container, such that on actuating the release means, the pressurized fluid is urged from the container into the chamber under its own head of pressure. In such cases, the release means may be the same integer as the means to cause the pressurized fluid to move into the chamber to engage with the particulate. A pressurized fluid container has the advantage in the dispenser according to the present invention of potentially providing a more rapid and/or stronger fluid discharge into the chamber. This may be desirable to de-aggregate particulate accommodated within the chamber if aggregated and to agitate it into turbulent flow to produce a mobile fluid comprising the particulate, especially if the particulate tends to clump fairly readily, on storage and/or in transit.

Amphiphilic carbohydrate compounds such as GCPQ are biocompatible polymers which can wrap around the incorporated drug to create a protective molecular envelope. Drugs, such as the preferred non-psychoactive cannabinoids in the invention, can then be loaded with high efficiency into the resulting stable nanoparticles.

The nanoparticle composition may be synthesised by common nanoparticle synthesis methods known in the art. This includes sol-gel methods, chemical vapor deposition (CVD), atomic layer deposition (ALD), molecular layer deposition (MLD), high pressure homogenisation and other microfluidization techniques, probe sonication, simply shaking the drug in the presence of the amphiphilic carbohydrate within aqueous media, milling, thin film production followed by hydration. Preferably the method comprises the dissolution of the cannabinoid into a solution of amphiphilic carbohydrate followed by evaporation to form a thin film, which is then further rehydrated. The ratio of non-psychoactive cannabinoid to amphiphilic carbohydrate compound is generally between 0.5:10 to 5:10 g g⁻¹. The ratio of cannabinoid to amphiphilic carbohydrate compound may be from 0.5:10 to 5:10 g g⁻¹ or from 0.5:10 to 4:10 g g⁻¹ or from 0.5:10 to 3:10 g g⁻¹ or from 0.5 to 10 to 2:10 g g⁻¹ or from 0.5 to 10 to 1:10 g g⁻¹ or from 1:10 to 5:10 g g⁻¹ or from 1:10 to 4:10 g g⁻¹ or from 1:10 to 3:10 g g⁻¹ or from 1 to 10 to 2:10 g g⁻¹ and the ratio may be 1:1 or 1:2 or 1:3 or 1:4 or 1:5 or 1:6 or 1:7 or 1:8 or 1:9 or 1:10 g g⁻¹ further preferably the ratio is 1:5 g g⁻¹.

In one preferred embodiment the weight ratio of cannabinoid to amphiphilic carbohydrate compound is above 1:0.5 and is preferably in the range 1:1 to 1:10, even more preferably 1:5 to 1:3. Such ratios are typically used with GCPQ as the amphiphilic carbohydrate compound, which has preferred palmitoylation and quaternisation levels as defined above. Accordingly in a further aspect of the invention there is provided a composition comprising GCPQ and a cannabinoid wherein the GCPQ has a palmitoylation level in the range 11-31 mole % and a quaternisation level in the range 8-17 mole %, and the weight ratio of GCPQ:cannabinoid is between 1:1 and 1:10. Preferably this composition is a pharmaceutical composition comprising the GCPQ and a non-psychoactive cannabinoid and one or more pharmaceutically acceptable excipients.

The resulting nanoparticle composition may be transformed into a nano-in-microparticles composition by further processing. For example, the nano-in-microparticles composition may be formed by spray-drying or lyophilisation of the nanoparticle composition. The nano-in-microparticle composition may also be formed by adding the nanoparticle composition to powders to form granules. The nano-in-microparticle composition may be in the form of a dry powder.

The nano-in-microparticle composition is particularly suitable for reliable and selective delivery of large micro-particles to the top of the nose, allowing the micro-particles to disintegrate into smaller particles and effectively penetrate mucus to facilitate mucosal uptake.

The nanoparticle and nano-in-microparticles compositions according to the invention are also capable of being stored for at least 30 days without experiencing significant degradation of their stability and characteristics, such as size and morphology. The formulations of the present invention should also be stable for extended periods of time as measured by the stability of the API in particular the cannabinoid over time. The formulations of the present invention have at least 50% or at least 60% or at least 70% or at least 80% or at least 85% or at least 90% or at least 95% or at least 99% recovery of the API after being stored 4 weeks at 4° C. or 25° C. The formulations of the present invention have at least 50% or at least 60% or at least 70% or at least 80% or at least 85% or at least 90% or at least 95% or at least 99% recovery of the API after being stored 4 weeks at 40° C. The formulations of the present invention have from about 50% to about 99% or from about 50% to 90% or from about 50% to about 80% or from about 50% to about 70% or from about 50% to 60% or from about 60% to about 99% or from about 60% to 90% or from about 60% to about 80% or from about 60% to about 70% or from about 70% to about 99% or from about 70% to 90% or from about 70% to about 80% or from about 80% to about 99% or from about 80% to about 90% or from about 90% to about 99% recovery of the API after being stored 4 weeks at 25° C. The formulations of the present invention have from about 50% to about 99% or from about 50% to 90% or from about 50% to about 80% or from about 50% to about 70% or from about 50% to 60% or from about 60% to about 99% or from about 60% to 90% or from about 60% to about 80% or from about 60% to about 70%, or from about 70% to about 99% or from about 70% to 90% or from about 70% to about 80% or from about 80% to about 99% or from about 80% to about 90% or from about 90% to about 99% recovery of the API after being stored 4 weeks at 40° C.

The composition of the invention may be further formulated to form a pharmaceutical composition which is particularly suited for intranasal delivery.

Additional ingredients that may be included in the pharmaceutical composition include tonicity enhancers, preservatives, solubilisers, non-toxic excipients, demulcents, sequestering agents, pH adjusting agents, co-solvents and viscosity building agents.

The pharmaceutical composition may be formulated as a powder or liquid dispersion. Delivery of the nasal spray may be accomplished by a spray device.

The dose can be determined on age, body weight, administration time, administration method, combination of drugs, the severity of the clinical condition or the actual condition for which a patient is undergoing therapy and other factors. While the daily doses may vary depending on the conditions and body weight of patients, the species or active ingredient, and administration route, in the case of oral use, the daily doses may be about 0.1 mg-10.0 g/person/day, or from 0.1 mg to 5.0 g/person/day or from 0.1 mg to 1.0 g/person/day or from 0.1 mg to 2.5 g/person/day or from 0.1 mg to 1.0 g/person/day or from 0.1 mg to 500 mg/person/day or from 0.5 mg to 5 g/person/day or from 0.5 mg to 2.5 g/person/day or from 0.5 mg to 1.0 g/person/day or from 0.5 mg to 0.5 g/person/day or from 0.5 mg to 100 mg/person/day or from or from 1 mg to 2.5 g/person/day or from 1 mg to 1.0 g/person/day or from 1 mg to 0.5 g/person/day or from 1 mg to 1.0 g/person/day or from 1 mg to 500 mg/person/day or from or from 10 mg to 2.5 g/person/day or from 10 mg to 1.0 g/person/day or from 10 mg to 0.5 g/person/day or from 10 mg to 0.1 g/person/day or from 10 mg to 50 mg/person/day or from 0.5-5000 mg/person/day or from 5-500 mg/person/day or from 10-250 mg/person/day or from 25-200 mg/person/day.

The invention will now be illustrated by the following Examples.

### EXAMPLES

### Example 1—Synthesis of GCPQ-CBD Nanoparticles

### Materials and Method

CBD-GCPQ nanoparticles were synthesised with two different concentration ratios: 1:5 and 1:10 CBD:GCPQ.

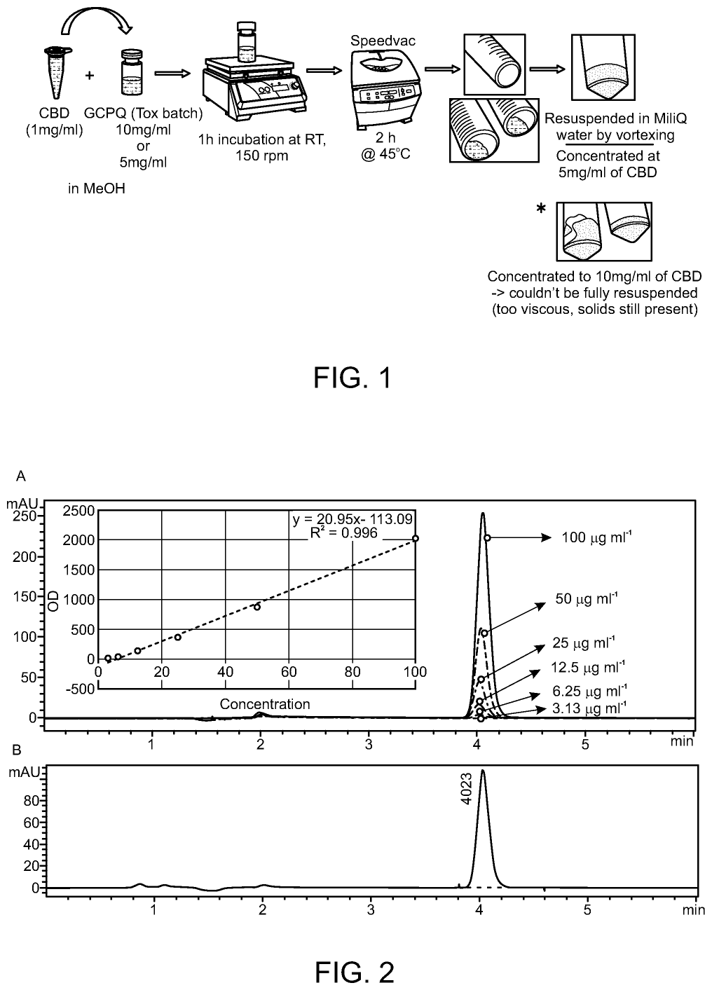

Cannabidiol (CBD>99%; THC Pharm) was dissolved in methanol at 1 mg mL⁻¹ for both ratios. Meanwhile, GCPQ GC_{17.5}P₁₈Q₁₃ was dissolved in methanol at 5 mg mL⁻¹ and 10 mg mL⁻¹. The solutions were then mixed together by adding the CBD solution to the GCPQ solution dropwise while swirling and subsequently vortexing for a few seconds. The mixture was incubated at room temperature for 1 h while shaking at 150 rpm. Next, the CBD-GCPQ formulation was evaporated using a speed-vacuum concentrator—for 2 h at 45° C. and the resulting thin films were rehydrated in Milli-Q water at 5 mg mL⁻¹ (1:5 ratio) and 10 mg mL⁻¹ (1:10 ratio). A schematic of the method is provided by FIG. 1.

### Characterization Methods

The synthesised CBD-GCPQ nanoparticles were then characterised for colloidal stability and CBD content quantification.

### Colloidal Stability:

Characterization was carried out using a Dynamic Light Scattering technique (DLS). The samples were diluted by 30× in Milli-Q water and the particle size, polydispersity and zeta potential parameters were measured on a Malvern Zetasizer Nano S/N using folded capillary zeta cell cuvettes. The protocol for size and zeta potential measurements included a 30 s equilibration time prior to data acquisition for each sample and measurements were taken in triplicate.

### CBD Quantification:

High-Performance Liquid Chromatography (HPLC) method for CBD detection and quantification in non-biological samples of MET-CBD formulations, was performed on an Agilent HPLC-UV system 1220 Infinity LC with a mobile phase of methanol:water 85:15% v/v. All samples submitted for HPLC were diluted by 100× in methanol. Data analysis was performed via Agilent Chemstation software.

### Results

The HPLC results for quantifying CBD generated a linear calibration curve within the range of 3 to 100 μg/mL (FIG. 2A) based on spiked known concentrations of CBD in methanol. Further, a well-defined single CBD peak in CBD-GCPQ formulations was also generated at a matching retention time of ˜4 minutes.

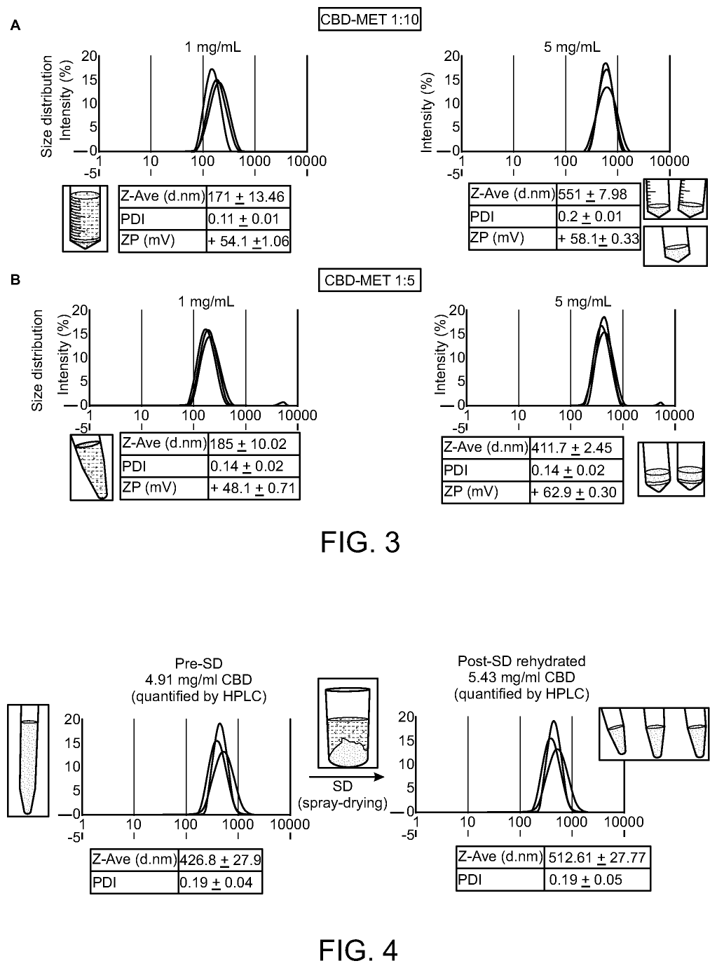

At 1 mg mL⁻¹ of CBD concentration equivalent, the formulations showed favourable colloidal stability parameters with sizes under 200 nm, a low polydispersity of ˜0.1 and a highly positive surface charge of around +50 mV (FIG. 3). For in vivo application higher CBD concentrations are required, thus 5-10 mg mL⁻¹ of CBD equivalent in the CBD-GCPQ nanoparticle formulations were tried. The 10 mg mL⁻¹ could not be achieved, as the formulation could not be fully dispersed at this high concentration, having high viscosity and solids remaining despite long and vigorous vortexing and bath sonication. This was particularly the case with the CBD, GCPQ 1:10 g g⁻¹ ratio, as this formulation had a high level of GCPQ and thus a higher solid content. CBD-GCPQ formulations were successfully formulated in in water at 5 mg mL⁻¹ CBD, for both CDB, GCPQ ratios (1:10 and 1:5 g g-1), producing colloidally stable nanoparticles, with monomodal particle population distributions which showed no signs of sedimentation or aggregation. CBD-GCPQ at a 1:5 g g⁻¹ ratio produced more favourable particle sizing parameters when compared to the CBD-GCPQ 1:10 g g⁻¹ ratio, i.e. nanoparticles of ˜412 nm (CBD-GCPQ=1:5 g g⁻¹) in size relative to 551 nm (CBD-GCPQ=1:10 g g-1) and slightly lower polydispersity index (PDI) of 0.14 (CBD-GCPQ=1:5 g g-1) relative to 0.2 (CBD-GCPQ=1:10 g g⁻¹).

### Example 2 MET-CBD Nano-In-Microparticle Powder

### Materials and Method

CBD-GCPQ nanoparticles were prepared according to the method in Example 1 from CBD (60 mg) and GCPQ (300 mg) at a ratio of 1:5 and then resuspended in 12 ml Milli-Q water prior to spray-drying. The nanoparticle dispersion was spray-dried to give CBD-GCPQ nano-in-microparticles (Büchi Nano Spray Dryer B290, Büchi Labortechnik AG, Switzerland) at the following settings: inlet temperature=180° C., outlet temperature=120° C., aspirator %=85%, pump %=5%, ultrasonic controller=1.8 at <50° C.).

### Characterization Methods

The synthesised CBD-GCPQ nanoparticles were then characterised for microparticle morphology and size analysis, colloidal stability, and CBD content quantification of the nanoparticles in both non-biological and biological samples. Some of the spray-dried CBD-GCPQ powders were stored at room temperature for 30 days, meanwhile other samples were stored at 4° C. for 30 days. At various time points (0, 7, 14 and 30 days) aliquots were taken to conduct characterizations.

### Microparticle Size:

The microparticle size of the spray-dried MET-CBD nano-in-microparticles was determined by laser scattering using a Malvern Mastersizer 3000. An aliquot of the powder (˜10 mg) was applied to the sample feeding tray. Air was used as the dispersion medium for the microparticles from the sample feeding tray to the sample cell. The microparticle size distribution was characterised using the D₁₀, D₅₀, and D₉₀ parameters.

### Microparticle Morphology:

The morphology of the microparticles was determined by Scanning Electron Microscopy (SEM) imaging. A strip of double-sided carbon tape was placed on an SEM stub. CBD-GCPQ powder was spread across the surface of the tape and compressed air was used to remove loose microparticles. Samples were coated with a 20 nm gold sputter before measurement. A Phenom Pro Benchtop SEM was used to generate SEM images of the samples.

### Colloidal Stability:

The samples were prepared and characterized for colloidal stability according to the method described in Example 1.

### CBD Quantification:

The samples were prepared and characterized for CBD quantification in non-biological samples according to the method described in Example 1.

### Results

The SEM characterization showed that the spray-dried particles had a largely spherical morphology compared to CBD powder alone, which had a more irregular morphology. The mean sizes of the particles (with the SEM error value) are provided in Table 1 below:

TABLE 1
Mean particle size, D₁₀, D₅₀, D₉₀ and % less
than 10 μm for CBD-GCPQ nano-in-microparticles

| | D₁₀(μm) | D₅₀ (μm) | D₉₀ (μm) | % <10 μm |
|---|---|---|---|---|
| Mean particle size | 14.59 ± | 22.09 ± | 34.37 ± | 7.51 ± |
| | 2.62 | 3.13 | 4.18 | 2.18 |

Rehydration of the spray-dried particles in water to 5.4 mg mL⁻¹ of CBD (analysed by HPLC) produced nanoparticles of ˜500 nm in size, which were a monomodal particle size distribution with a low polydispersity (PDI=<0.2). The size of rehydrated nanoparticles appeared to increase slightly compared to the nanoparticles prior to spray-drying. However, this can most likely be attributed to the difference in the actual measured CBD concentration and consequently the amount of GCPQ in both CBD-GCPQ formulations, namely 5.43 mg mL⁻¹ in the post spray-dry formulation compared to the original 4.93 mg mL⁻¹, as shown by FIG. 4. This may have given rise to the slightly higher particle size, since this is a typical trend noticed with concentrated formulations. The polydispersity pre- and post spray-drying was maintained at approximately 0.19 (FIG. 4).

Upon storage for 30 days at both room temperature and 4° C., the spray-dried CBD-GCPQ microparticles retained their spherical morphology throughout the several time points measured, namely day 0, 7, 14 and 30 days. All size parameters of spray-dried CBD-GCPQ powders stored in room temperature and 4° C. for 0, 7, 14 and 30 days are provided in Tables 2 and 3 below.

TABLE 2
Size distribution of nano-in-microparticles stored
at room temperature at 0, 7, 14, and 30 days.

| | D₁₀(μm) | D₅₀ (μm) | D₉₀ (μm) | % <10 μm |
|---|---|---|---|---|
| Day 0 | 18.71 ± 5.06 | 41.56 ± 7.69 | 67.16 ± 13.08 | 4.10 ± 3.72 |
| Day 7 | 11.94 ± 2.82 | 28.48 ± 7.98 | 57.99 ± 20.88 | 13.58 ± 5.06 |
| Day 14 | 18.33 ± 2.70 | 40.57 ± 1.87 | 67.58 ± 1.98 | 4.67 ± 5.06 |
| Day 30 | 12.13 ± 2.49 | 22.41 ± 5.43 | 42.56 ± 14.14 | 13.04 ± 5.06 |

TABLE 3
Size distribution of nano-in-microparticles
stored at 4° C. at 0, 7, 14, and 30 days.

| | D₁₀(μm) | D₅₀ (μm) | D₉₀ (μm) | % <10 μm |
|---|---|---|---|---|
| Day 0 | 18.71 ± 5.06 | 41.56 ± 7.69 | 67.16 ± 13.08 | 4.10 ± 3.72 |
| Day 7 | 13.06 ± 2.94 | 30.94 ± 4.58 | 71.56 ± 12.82 | 10.12 ± 4.21 |
| Day 14 | 21.85 ± 4.14 | 33.07 ± 6.03 | 49.64 ± 7.97 | 3.29 ± 3.28 |
| Day 30 | 10.42 ± 1.83 | 23.69 ± 5.85 | 43.01 ± 9.45 | 15.44 ± 3.12 |

Median size volume distribution (Do) and % of particles below 10 mm did not show statistically significant differences throughout 30-day storage at either of the temperatures.

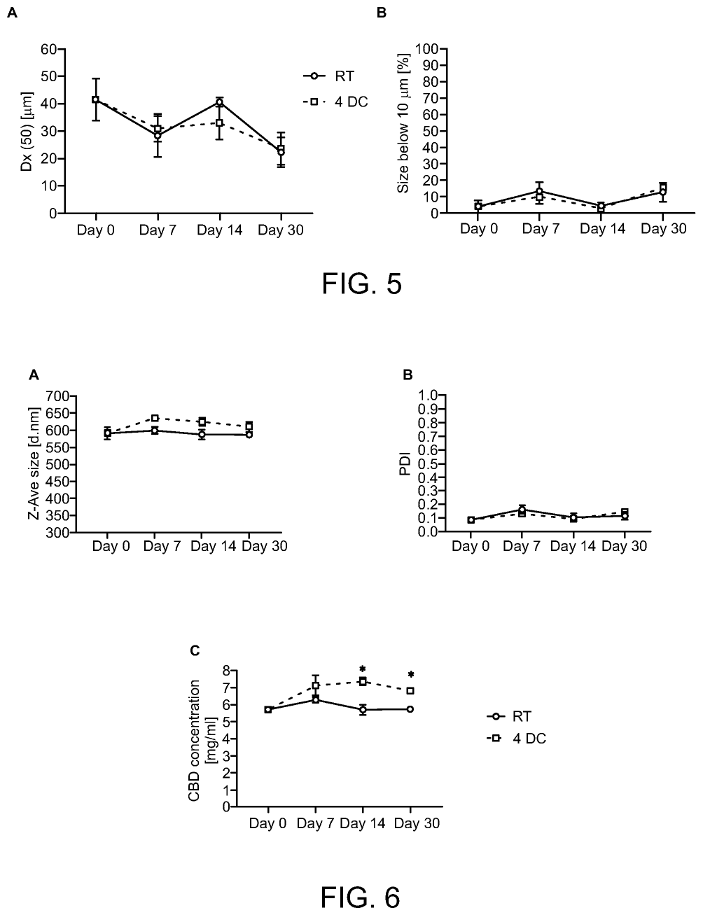

Spray-dried CBD-GCPQ powder was then rehydrated to 5 mg mL⁻¹ CBD equivalent and nanoparticle colloidal stability, as well as CBD concentrations, were analysed and compared to the day 0 parameters. No statistically significant differences in particle size were observed across the time period, as shown by FIG. 5. Similarly, no significant differences in polydispersity and zeta potential were observed for up to 30 days at either room temperature of 4° C. (FIGS. 6A and B). No CBD degradation within the CBD-GCPQ nanoparticle formulation was observed for up to 30 days at either of the temperatures (FIG. 6C), although refrigerated powder surprisingly yielded significantly higher CBD concentrations over time (at day 14 and 30). In contrast, Mazzetti et al. in Scientific Reports from NatureResearch (2020) 10:3697 reported an average degradation by 13% in 30 days among commercially available CBD oil-based E-liquids (electronic cigarette liquid) at room temperature when exposed to light. Thus, the present invention demonstrates clear stability advantages over the art by forming stable and efficacious non-psychoactive cannabinoid-amphiphilic carbohydrate compositions.

### Example 3—In Vivo Pharmacokinetics Study in Rats Intranasally Dosed with MET-CBD

### Materials and Methods

### Animals:

Male Sprague Dawley rats (Charles River, UK) were housed five per cage in an air-conditioned unit (20-22° C., 50-60% relative humidity) and allowed free access to standard rodent chow and water. Lighting was controlled on a twelve-hour cycle (on at 07.00 h and out at 19.00 h). Animals were habituated for 7 days prior to experimentation and acclimatized to the procedure room for 1 h prior to testing. Rats weighed 200-230 g prior to dosing and were stratified to have matching average weights across test groups.

### CBD-GCPQ Formulation for In Vivo Application and Intranasal Dosing:

Prior to dosing the rats, the CBD-GCPQ formulation according to Example 2 was dispersed in Milli-Q water at 5 mg mL⁻¹ of CBD. The pH of the formulation was measured and adjusted to pH=5.5-5.6 upon the addition of NaOH. The rats were stratified into 5 test groups containing 6 animals each. Four groups were dosed with CBD-GCPQ for four post-dosing termination timepoints (10 min., 30 min., 1 h, 2 h) and one group was left non-dosed. CBD-GCPQ was dosed intranasally (at 2 mg kg⁻¹ of CBD equivalent) using Smiths Medical Portex fine bore polythene tubing of 0.28/0.61 mm inner/outer diameter of 15 mm length attached to 0.3 mL insulin syringe with 30 G needle. Animals were anesthetised by inhalation of isoflurane (˜4%) for a few minutes before the dosing.

### Tissue Extraction and Blood Collection:

At timepoints of 10 min., 30 min., 1 h and 2 h, the post-dosing rats were sacrificed by carbon dioxide asphyxiation and deaths were confirmed by cervical dislocation. Blood samples, from which plasma was later sourced, were taken immediately after the termination via cardiac puncture and collected into K3EDTA anti-coagulant microtubes.

Plasma was separated from blood cells by centrifugation, aspirated into 1.5 mL Eppendorf microtubes and stored frozen at −50° C. until LC-MS analysis could be performed.

Brains were extracted and snap-frozen in liquid nitrogen and then stored frozen at −80° C. until LC-MS analysis could be performed. Brain homogenate and plasma then served as the biological matrices for the compound extraction and LC-MS analysis.

### Characterization

The synthesised CBD-GCPQ nanoparticles were then characterized for CBD content in biological samples (brain and plasma from the rats).

CBD quantification:

The CBD content in the biological samples was quantified using liquid chromatography-mass spectroscopy (LC-MS). The instrument Agilent 6400 Series Triple Quad was used to measure the CBD and its internal standard, deuterated CBD (CBD-d3, Molecular weight=318 Da) in brain and plasma matrices. First, non-biological samples containing known concentrations of spiked target compound CBD and the internal standard CBD-d3 were analysed via LC-MS scans. The process involved a three-step workflow: 1) a full tandem spectroscopy (MS2) scan to identify the precursor ions for both compounds; 2) a Product Ion scan was run, wherein the selected precursor ions are fragmented to form and identify product ions; and 3) a multiple reaction monitoring (MRM) scanning mode was run on the CBD target compound based on the selected product ions to quantify the target compounds in the sample. The binary pump included a mobile phase of water:methanol at 95:5% v/v at 0 minutes followed by 15:85% v/v at 2 minutes. The source parameters were set to have a gas temperature of 300-350° C.

LC-MS methods typically lose sensitivity once analyte quantification in biological matrices is introduced; commonly known as the “matrix effect”. Therefore, before analysing the brains and plasma from the in vivo study, the MRM method was optimised by varying the mobile phase composition, namely by using different polar and organic solvents and an isocratic versus gradient composition.

### Results

### CBD Quantification for In Vivo Preclinical Study:

The CBD quantification method revealed a well-defined peak for CBD at a retention time of 1 minute via the isocratic method for the non-biological sample. A well-defined peak was also observed at 4.4 min via the gradient method for the biological matrices.

The first MS2 scan identified the most abundant precursor CBD ion at a mass-to-charge (m/z) ratio of 315.2 and CBD-d3 ion at m/z of 318.2, which is consistent with the literature values provided in McRae, G. et al., (Quantitative determination and validation of 17 cannabinoids in cannabis and hemp using liquid chromatography-tandem mass spectrometry. Analytical and Bioanalytical Chemistry, 412:7381-7393, 2020). This CBD precursor ion was then selected for the fragmentation to product ions in the second step of the workflow. The product ion scan showed the CBD precursor ion was completely broken down to product ions. The CBD product ion at m/z=193 and CBD-d3 product ion at m/z=196.3 were also confirmed against McRae G et al. and selected for the third step of the workflow.

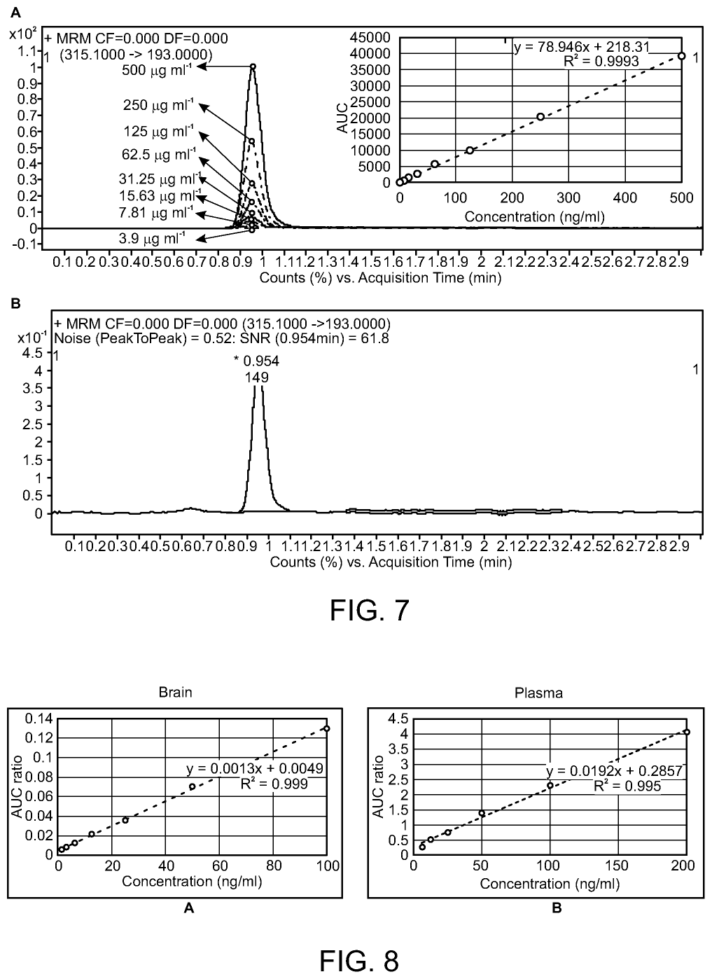

Based on the mass-to-charge ratio identified in the MS2 and Product Ion scans, the MRM method for CBD and CBD-d3 quantification was set up and a calibration curve in a non-biological sample was generated with very good linearity within the quantification range of 1-500 ng/mL, as shown by FIG. 7. A clearly defined CBD peak in the MRM scans confirmed the accuracy of the LC-MS quantification method and high sensitivity of the method, with a lower limit of quantification (LLOQ) of at least 1 ng/mL.

Calibration curves were generated in the brain tissue and plasma matrices with good linearity within respective quantification ranges of 1-100 ng/mL with R²=0.999 for brain and 3-200 ng/mL with R²=0.995 for plasma, as shown by FIG. 8. The LC-MS method yielded high sensitivity of CBD quantification, with LLOQ of 1 ng/mL and 3 ng/mL in brain and plasma matrices, respectively.

### Colloidal Stability, pH and Size Distribution:

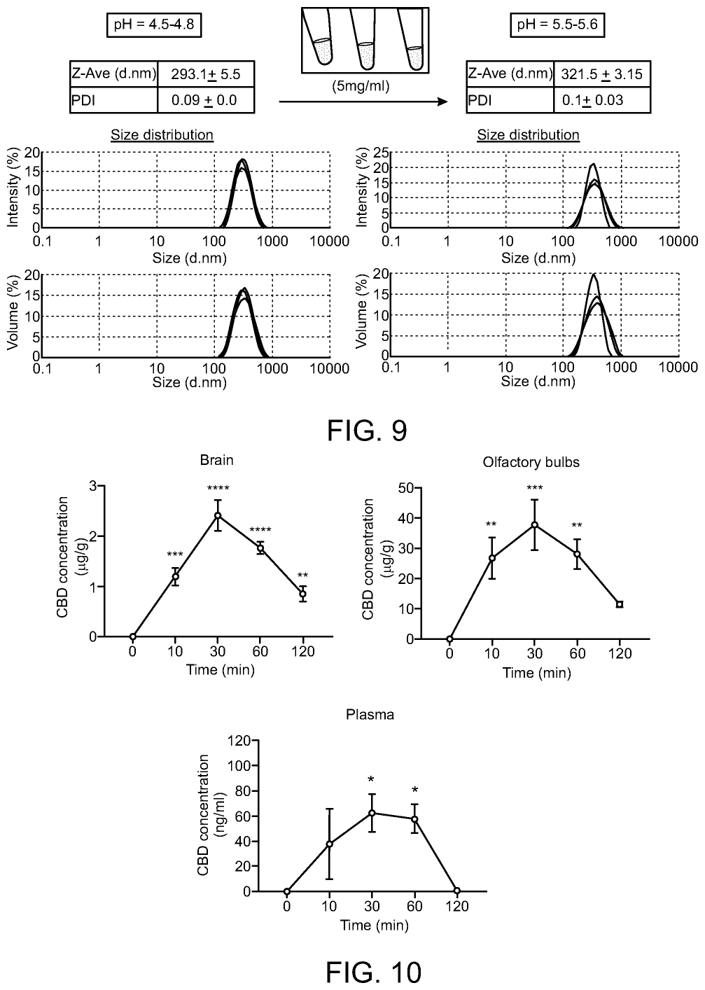

Prior to dosing the rats, the 5 mgmL⁻¹ nanoparticles were characterised for colloidal stability and pH. The nanoparticles formed a monodisperse (PDI<0.1) nanoparticle population sized below 300 nm, with a pH between 4.5-4.8. This pH is lower than the physiological pH in the nasal cavity, which according to England et al. (Nasal pH measurement: a reliable and repeatable parameter. Clin Otolaryngol Allied Sci, 24 (1):67-8, 1999) is between 5.5-5.6. As this can cause animal discomfort and undesired reactions like sneezing, the pH was adjusted to this range prior to dosing. The pH increase had a very minimal effect on the colloidal stability of the nanoparticles; the nanoparticle size was merely increased slightly to approximately 320 nm and the polydispersity index was still around 0.1. FIG. 9 compares the size distribution and polydispersity before and after pH adjustment.

### Intranasal Administration In Vivo Pharmacokinetics Study:

Overall, the intranasal administration of CBD-GCPQ was very well tolerated by the dosed animals. No sneezing, distress or adverse reactions were reported; the rats appeared to be calm and relaxed. As reported by Galaj et al. (Possible Receptor Mechanisms Underlying Cannabidiol Effects on Addictive-like Behaviors in Experimental Animals. Int. J. Mol. Sci., 22(1), 134, 2021) CBD can bind to certain receptors in the brain that release serotonin and in consequence produce the feeling of well-being, whereas THC induces psychostimulant effects and may induce addictive behaviours. Therefore, the observed rats' calm behaviour following dosing is indicative that the CBD reached the brain fairly rapidly.

FIG. 10 shows the CBD levels in the brain, olfactory bulbs and plasma of the tested rats administered 2 mg kg⁻¹ CBD at 0 minutes (non-dosed control), 10 minutes, 30 minutes, 60 minutes and 120 minutes. These results confirm that within 10 minutes of dosing, significant levels of CBD were present in the brain, olfactory bulbs and plasma. A maximum concentration was reached at 30 minutes for all profiles, with a gradual decrease in concentration after 60 minutes. Even 120 min post-dose, there were still some levels of CBD in the brain and olfactory bulbs. In the plasma, the CBD max concentration was 40-fold lower compared to the brain, suggesting very low systemic exposure following intranasal administration. These results have outperformed CBD levels reported in the literature. For example, Hozek et al. (Pharmacokinetic and behavioural profile of THC, CBD, and THC+CBD combination after pulmonary, oral, and subcutaneous administration in rats and confirmation of conversion in vivo of CBD to THC. European Neuropsychopharmacology, 27, 1223-1237, 2017) orally pulmonary dosed 10 mg kg⁻¹ and 20 mg kg⁻¹ respectively and achieved only ˜200-300 ng g⁻¹ of CBD levels in the brain, which is significantly lower than the 2,400 ng g⁻¹ reported in the present study (as shown by FIG. 10A). Thus, the present intranasal method achieved a 10-fold higher CBD concentration than the art, despite using a much lower dosage quantity of 2 mg kg⁻¹. Therefore, the present invention permits a lower clinical dose of the amphiphilic carbohydrate-non-psychoactive cannabinoid composition.

### Example 4: Generation and Characterisation of Further Compositions

The aim of this Example was to generate compositions comprising GCPQs with different amounts of palmitoylation (d unit in formula I) and quaternisation (b unit). Different ratios of cannabinoid to GCPQ were also tested.

### Materials and Methods

CBD (THC Pharm, UK) and GCPQ formulations were prepared by a thin-film evaporation method. Several GCPQ polymers with different percentages of mole % palmitoylation (% P) and mole % quaternary ammonium groups (% Q) were explored, with the level of palmitoylation and quaternary ammonium groups depicted in the polymer identification labels: GCP11Q11; GCP19Q8; GCP20Q13; GCP20Q17; GCP31Q13. In these depictions, by way of example GCP11Q11 refers to a polymer with 11 mole % palmitoylation and 11 mole % quaternary ammonium groups.

CBD and MET were dissolved in a round bottom flask at 1:0.5, 1:1, 1:3, 1:5 and 1:10 CBD to MET ratio in MeOH at a final CBD concentration of 1 mg mL⁻¹. The MeOH mixture was left under stirring for 1 h at room temperature. Subsequently, a thin film was obtained by rotary evaporation under vacuum in a 45° C. water bath and this was rehydrated with milliQ water to a final CBD concentration of 5 mg mL⁻¹. The dispersion was then sonicated in a sonicator bath at 30° C. for 10 minutes.

Dynamic Light Scattering and Zeta Potential Dynamic Light Scattering (DLS) and zeta (ζ) potential measurements were carried out with a Zeta-sizer Ultra (Malvern Instruments Ltd). Dispersions were diluted 30-fold in milliQ water and equilibrated at 25° C. for 120 seconds prior to measurement. Measurements were collected in triplicates. The software uses the CONTIN analysis to derive the intensity distribution and the cumulant analysis to obtain the hydrodynamic diameter (DH) and the polydispersity index (PI).
CBD Content Quantification by High-Pressure Liquid Chromatography (HPLC) CBD content in the MET-CBD formulations was quantified by HPLC. Settings used are shown below:

### HPLC Method

Agilent HPLC-UV system 1260 Infinity II
Column=Agilent Eclipse Plus C-18 4.6 mm×150
mm, 3.5 μm
Mobile phase=Methanol:water (85:15%, v/v)
Detection wavelength=220 nm
Flow=1.0 mL min-1
Injection volume=10 μL
Run time=6 min.
Retention time=˜4 min.

A linear (R²=0.9996) calibration curve was generated within a concentration range of 1 to 150 μg/mL of CBD in methanol. A well-defined single CBD peak was found in MET-CBD formulations at the same retention time as CBD alone (˜4 min). All samples submitted for HPLC were diluted 100× in methanol. Data analysis was performed via Agilent Chemstation software.

### Results

The prepared formulations were characterised in terms of size, polydispersity index (PI), ζ-potential and CBD content (%). The stability of these formulations depended on the type of polymer used, in terms of % P and % Q, and the CBD, polymer ratio. The stability was followed over time up to 8 hours or until formation of a suspension/precipitate. In all cases a precipitate could be observed after 24 hours.

TABLE 4
Summary of results (Z-Average; PI, ζ-potential and CBD content) for CBD:GCP11Q11 over time.
CBD:GCP11Q11

| | | | | | |
|---|---|---|---|---|---|
| Hours | 0 | 2 | 4 | 6 | 8 |

1:0.5 ratio

| | | | | | |
|---|---|---|---|---|---|
| Z-Average (nm) | 655 ± 54.6 | 792 ± 314.8 | — | — | — |
| PI | 0.267 ± 0.203 | 0.528 ± 0.239 | — | — | — |
| ζ-potential (mV) | +71 ± 4.06 | — | — | — | — |
| CBD content (%) | 105.78 ± 0.05 | — | 54.47 ± 0.06 | 48.28 ± 0.03 | — |
| Appearance | Milky/Stable | Sedimentation | Precipitate | Precipitate | Precipitate |
| | suspension | | | | |

1:1 ratio

| | | | | | |
|---|---|---|---|---|---|
| Z-Average (nm) | 467 ± 12.9 | 513 ± 8.8 | 562 ± 4.4 | 562 ± 4.4 | 596 ± 10.8 |
| PI | 0.067 ± 0.006 | 0.031 ± 0.019 | 0.084 ± 0.041 | 0.099 ± 0.069 | 0.207 ± 0.052 |
| ζ -potential (mV) | +66 ± 0.87 | — | — | — | — |
| CBD content (%) | 112.48 ± 0.06 | — | 106.64 ± 0.10 | 109.12 ± 0.17 | — |
| Appearance | Milky/Stable | Milky/Stable | Milky/Stable | Milky/Stable | Milky/Stable |
| | suspension | suspension | suspension | suspension | suspension |

1:3 ratio

| | | | | | |
|---|---|---|---|---|---|
| Z-Average (nm) | 436 ± 5.9 | 537 ± 3.1 | 640 ± 4.9 | 665 ± 2.9 | 696 ± 14.5 |
| PI | 0.026 ± 0.038 | 0.058 ± 0.037 | 0.110 ± 0.052 | 0.025 ± 0.007 | 0.080 ± 0.036 |
| ζ -potential (mV) | +72 ± 0.75 | — | — | — | — |
| CBD content (%) | 110.65 ± 0.11 | — | 110.50 ± 0.04 | 109.05 ± 0.07 | — |
| Appearance | Milky/Stable | Milky/Stable | Milky/Stable | Milky/Stable | Milky/Stable |
| | suspension | suspension | suspension | suspension | suspension |

1:5 ratio

| | | | | | |
|---|---|---|---|---|---|
| Z-Average (nm) | 394 ± 1.8 | 480 ± 6.2 | 537 ± 9.30 | 556 ± 5.1 | 568 ± 8.51 |
| PI | 0.097 ± 0.040 | 0.094 ± 0.007 | 0.054 ± 0.057 | 0.070 ± 0.045 | 0.104 ± 0.061 |
| ζ -potential (mV) | +72 ± 0.52 | — | — | — | — |
| CBD content (%) | 105.13 ± 0.04 | — | 103.02 ± 0.07 | 104.73 ± 0.44 | — |
| Appearance | Milky/Stable | Milky/Stable | Milky/Stable | Milky/Stable | Milky/Stable |
| | suspension | suspension | suspension | suspension | suspension |

1:10 ratio

| | | | | | |
|---|---|---|---|---|---|
| Z-Average (nm) | 489 ± 9.8 | 644 ± 32.7 | 762 ± 24.8 | 802 ± 19.6 | 821 ± 24.0 |
| PI | 0.063 ± 0.051 | 0.037 ± 0.028 | 0.158 ± 0.124 | 0.114 ± 0.069 | 0.278 ± 0.115 |
| ζ -potential (mV) | +73 ± 0.82 | — | — | — | — |
| CBD content (%) | 108.64 ± 0.19 | — | 101.73 ± 0.09 | 106.16 ± 0.32 | — |
| Appearance | Milky/Stable | Milky/Stable | Milky/Stable | Milky/Stable | Milky/Stable |
| | suspension | suspension | suspension | suspension | suspension |

TABLE 5
Summary of results (Z-Average; PI, ζ-potential and CBD content) for CBD:GCP19Q8 over time (MW 12 kDa)
CBD:GCP19Q8

| | | | | | |
|---|---|---|---|---|---|
| Hours | 0 | 2 | 4 | 6 | 8 |

1:0.5 ratio

| | | | | | |
|---|---|---|---|---|---|
| Z-Average (nm) | 830 ± 116.3 | 1029 ± 59 | — | — | — |
| PI | 0.424 ± 0.244 | 0.497 ± 0.08 | — | — | — |
| ζ-potential (mV) | +80 ± 0.85 | — | — | — | — |
| CBD content (%) | 114.49 ± 0.03 | — | 94.75 ± 0.01 | 89.82 ± 0.18 | — |
| Appearance | Milky/Stable | Sedimentation | Precipitate | Precipitate | Precipitate |
| | suspension | | | | |

1:1 ratio

| | | | | | |
|---|---|---|---|---|---|
| Z-Average (nm) | 342 ± 2.3 | 369 ± 11.2 | 388 ± 13.9 | 393 ± 11.5 | — |
| PI | 0.113 ± 0.05 | 0.148 ± 0.024 | 0.203 ± 0.118 | 0.175 ± 0.05 | — |
| ζ -potential (mV) | +70 ± 2.19 | — | — | — | — |
| CBD content (%) | 115.28 ± 0.11 | — | 113.64 ± 0.04 | 110.69 ± 0.06 | — |
| Appearance | Milky/Stable | Milky/Stable | Milky/Stable | Milky/Stable | Precipitate |
| | suspension | suspension | suspension | suspension | |

1:3 ratio

| | | | | | |
|---|---|---|---|---|---|
| Z-Average (nm) | 383 ± 3.8 | 646 ± 7.68 | 707 ± 26.6 | 854 ± 60.3 | — |
| PI | 0.122 ± 0.03 | 0.080 ± 0.012 | 0.085 ± 0.100 | 0.160 ± 0.152 | — |
| ζ -potential (mV) | +78 ± 0.69 | — | — | — | — |
| CBD content (%) | 102.99 ± 0.18 | — | 106.23 ± 0.19 | 102.86 ± 0.03 | — |
| Appearance | Milky/Stable | Milky/Stable | Milky/Stable | Milky/Stable | Precipitate |
| | suspension | suspension | suspension | suspension | |

1:5 ratio

| | | | | | |
|---|---|---|---|---|---|
| Z-Average (nm) | 439 ± 11.7 | 655 ± 34.3 | 887 ± 60.21 | 1184 ± 59 | — |
| PI | 0.100 ± 0.06 | 0.085 ± 0.074 | 0.092 ± 0.075 | 0.179 ± 0.045 | — |
| ζ -potential (mV) | +81 ± 1.09 | — | — | — | — |
| CBD content (%) | 108.30 ± 0.20 | — | 109.69 ± 0.24 | −107.46 ± 0.11 | — |
| Appearance | Milky/Stable | Milky/Stable | Milky/Stable | Milky/Stable | Precipitate |
| | suspension | suspension | suspension | suspension | |

1:10 ratio

| | | | | | |
|---|---|---|---|---|---|
| Z-Average (nm) | 447 ± 5.6 | 663 ± 8.5 | 800 ± 8.0 | 858 ± 51.9 | 985 ± 102.7 |
| PI | 0.051 ± 0.051 | 0.073 ± 0.054 | 0.038 ± 0.008 | 0.097 ± 0.066 | 0.144 ± 0.062 |
| ζ -potential (mV) | +82 ± 0.59 | — | — | — | — |
| CBD content (%) | 111.30 ± 0.21 | — | 109.09 ± 0.02 | 102.31 ± 0.11 | — |
| Appearance | Milky/Stable | Milky/Stable | Milky/Stable | Milky/Stable | Milky/Stable |
| | suspension | suspension | suspension | suspension | suspension |

TABLE 6
Summary of results (Z-Average; PI, ζ-potential and CBD content) for CBD:GCP20Q13 over time.
CBD:GCP20Q13

| | | | | | |
|---|---|---|---|---|---|
| Hours | 0 | 2 | 4 | 6 | 8 |

1:0.5 ratio

| | | | | | |
|---|---|---|---|---|---|
| Z-Average (nm) | 529 ± 15.9 | 720 ± 26.7 | — | — | — |
| PI | 0.183 ± 0.05 | 0.259 ± 0.125 | — | — | — |
| ζ-potential (mV) | +77.65 ± 1.72 | — | — | — | — |
| CBD content (%) | 77 ± 0.18 | — | 71 ± 0.10 | 72 ± 0.22 | — |
| Appearance | Milky/Stable | Sedimentation | Precipitate | Precipitate | Precipitate |
| | suspension | | | | |

1:1 ratio

| | | | | | |
|---|---|---|---|---|---|
| Z-Average (nm) | 306 ± 2.6 | 347 ± 0.3 | 373 ± 7.8 | | — |
| PI | 0.114 ± 0.08 | 0.167 ± 0.03 | 0.239 ± 0.021 | | — |
| ζ -potential (mV) | +71 ± 0.35 | — | — | — | — |
| CBD content (%) | 111.52 ± 0.08 | — | 107.35 ± 0.19 | 81.33 ± 0.10 | — |
| Appearance | Milky/Stable | Milky/Stable | Milky/Stable | Sedimentation | Precipitate |
| | suspension | suspension | suspension | | |

1:3 ratio

| | | | | | |
|---|---|---|---|---|---|
| Z-Average (nm) | 395 ± 4.5 | 579 ± 13.5 | 857 ± 21.1 | 1115 ± 202 | — |
| PI | 0.093 ± 0.032 | 0.058 ± 0.040 | 0.083 ± 0.027 | 0.455 ± 0.328 | — |
| ζ -potential (mV) | +81 ± 0.59 | — | — | — | — |
| CBD content (%) | 112.14 ± 0.02 | — | 107.11 ± 0.09 | 104.82 ± 0.09 | — |
| Appearance | Milky/Stable | Milky/Stable | Milky/Stable | Milky/Stable | Precipitate |
| | suspension | suspension | suspension | suspension | |

1:5 ratio

| | | | | | |
|---|---|---|---|---|---|
| Z-Average (nm) | 410 ± 5.2 | 646 ± 5.6 | 810 ± 13.2 | 190 ± 16.7¹ | — |
| PI | 0.075 ± 0.028 | 0.066 ± 0.031 | 0.079 ± 0.011 | 0.348 ± 0.046 | — |
| ζ -potential (mV) | +72. ± 1.17 | — | — | — | — |
| CBD content (%) | 103.15 ± 0.14 | — | 100.70 ± 0.10 | 110.69 ± 0.06 | — |
| Appearance | Milky/Stable | Milky/Stable | Milky/Stable | Milky/Stable | Precipitate |
| | suspension | suspension | suspension | suspension | |

1:10 ratio

| | | | | | |
|---|---|---|---|---|---|
| Z-Average (nm) | 417 ± 4.1 | 634 ± 6.5 | 771 ± 7.1 | 834 ± 45.9 | 734 ± 68.7 |
| PI | 0.049 ± 0.026 | 0.061 ± 0.024 | 0.142 ± 0.097 | 0.092 ± 0.092 | 0.080 ± 0.04 |
| ζ-potential (mV) | +84 ± 2.21 | — | — | — | — |
| CBD content (%) | 96.47 ± 0.09 | — | 99.13 ± 0.09 | 98.61 ± 0.23 | — |
| Appearance | Milky/Stable | Milky/Stable | Milky/Stable | Milky/Stable | Milky/Stable |
| | suspension | suspension | suspension | suspension | suspension |

¹Multimodal size distribution.

TABLE 7
Summary of results (Z-Average; Pl, ζ-potential and CBD
content) for CBD:GCP20Q17 over time (MW = 11.9 kDa).
CBD:GCP20Q17

| | | | | | |
|---|---|---|---|---|---|
| Hours | 0 | 2 | 4 | 6 | 8 |

1:0.5 ratio

| | | | | | |
|---|---|---|---|---|---|
| Z-Average (nm) | 630 ± 8.3 | 785 ± 81.8 | | | |
| PI | 0.109 ± 0.142 | 0.177 ± 0.076 | | | |
| ζ-potential (mV) | +80 ± 2.17 | — | — | — | — |
| CBD content (%) | 101.25 ± 0.07 | — | 87.84 ± 0.03 | 83.70 ± 0.02 | — |
| Appearance | Milky/Stable | Milky/Stable | Sedimentation | Sedimentation | |
| | suspension | suspension | | | |

1:1 ratio

| | | | | | |
|---|---|---|---|---|---|
| Z-Average (nm) | 351 ± 5.8 | 484 ± 3.5 | 561 ± 33.6 | 574 ± 9.1 | 617 ± 36.4 |
| PI | 0.165 ± 0.023 | 0.224 ± 0.013 | 0.217 ± 0.120 | 0.157 ± 0.054 | 0.162 ± 0.132 |
| ζ -potential (mV) | +72 ± 3.61 | — | — | — | — |
| CBD content (%) | 114.28 ± 1.04 | — | 108.18 ± 0.08 | 110.36 ± 0.06 | — |
| Appearance | Milky/Stable | Milky/Stable | Milky/Stable | Milky/Stable | Milky/Stable |
| | suspension | suspension | suspension | suspension | suspension |

1:3 ratio

| | | | | | |
|---|---|---|---|---|---|
| Z-Average (nm) | 430 ± 6.8 | 607 ± 4.2 | 792 ± 5.7 | 859 ± 96.6 | 860 ± 102 |
| PI | 0.042 ± 0.054 | 0.026 ± 0.018 | 0.073 ± 0.037 | 0.522 ± 0.411 | 0.208 ± 0.201 |
| ζ -potential (mV) | +82 ± 0.69 | — | — | — | — |
| CBD content (%) | 110.31 ± 0.14 | — | 107.60 ± 0.26 | 108.17 ± 0.12 | — |
| Appearance | Milky/Stable | Milky/Stable | Milky/Stable | Milky/Stable | Milky/Stable |
| | suspension | suspension | suspension | suspension | suspension |

1:5 ratio

| | | | | | |
|---|---|---|---|---|---|
| Z-Average (nm) | 418 ± 2.3 | 691 ± 4.6 | 829 ± 34.1 | 846 ± 88.9 | 839 ± 36.5 |
| PI | 0.095 ± 0.023 | 0.114 ± 0.075 | 0.063 ± 0.018 | 0.286 ± 0.385 | 0.164 ± 0.027 |
| ζ -potential (mV) | +2 ± 2.54 | — | — | — | — |
| CBD content (%) | 112.53 ± 0.06 | — | 107.93 ± 0.22 | 107.90 ± 0.03 | — |
| Appearance | Milky/Stable | Milky/Stable | Milky/Stable | Milky/Stable | Milky/Stable |
| | suspension | suspension | suspension | suspension | suspension |

1:10 ratio

| | | | | | |
|---|---|---|---|---|---|
| Z-Average (nm) | 484 ± 3.6 | 767 ± 2.6 | 872 ± 47.1 | 874 ± 64.0 | 996 ± 166.5 |
| PI | 0.029 ± 0.039 | 0.301 ± 0.135 | 0.120 ± 0.104 | 0.055 ± 0.045 | 0.643 ± 0.371 |
| ζ -potential (mV) | +84 ± 1.73 | — | — | — | — |
| CBD content (%) | 116.96 ± 0.22 | — | 106.21 ± 0.10 | 112.95 ± 0.13 | — |
| Appearance | Milky/Stable | Milky/Stable | Milky/Stable | Milky/Stable | Milky/Stable |
| | suspension | suspension | suspension | suspension | suspension |

TABLE 8
Summary of results (Z-Average; PI, ζ-potential and CBD content) for CBD:GCP31Q13 over time.
CBD:GCP31Q13

| | | | | | |
|---|---|---|---|---|---|
| Hours | 0 | 2 | 4 | 6 | 8 |

1:0.5 ratio

| | | | | | |
|---|---|---|---|---|---|
| Z-Average (nm) | 522 ± 9.5 | 583 ± 53.2 | — | — | — |
| PI | 0.131 ± 0.152 | 0.301 ± 0.190 | — | — | — |
| ζ-potential (mV) | +72 ± 1.53 | — | — | — | — |
| CBD content (%) | 117.07 ± 0.08 | — | 102.75 ± 0.26 | 64.87 ± 0.13 | — |
| Appearance | Milky/Stable | Milky/Stable | Sedimentation | Precipitate | Precipitate |
| | suspension | suspension | | | |

1:1 ratio

| | | | | | |
|---|---|---|---|---|---|
| Z-Average (nm) | 310 ± 7.9 | 379 ± 36.8 | 414 ± 2.3 | 480 ± 9.7 | — |
| PI | 0.214 ± 0.044 | 0.279 ± 0.083 | 0.189 ± 0.051 | 0.246 ± 0.166 | — |
| ζ -potential (mV) | +74 ± 1.12 | — | — | — | — |
| CBD content (%) | 117.92 ± 0.05 | — | 116.58 ± 0.10 | 112.96 ± 0.11 | — |
| Appearance | Milky/Stable | Milky/Stable | Milky/Stable | Milky/Stable | Sedimentation |
| | suspension | suspension | suspension | suspension | |

1:3 ratio

| | | | | | |
|---|---|---|---|---|---|
| Z-Average (nm) | 491 ± 1.7 | 606 ± 37.4 | 1020 ± 101 | — | — |
| PI | 0.031 ± 0.018 | 0.110 ± 0.024 | 0.169 ± 0.144 | — | — |
| ζ -potential (mV) | +82 ± 2.92 | — | — | — | — |
| CBD content (%) | 113.67 ± 0.14 | — | 117.64 ± 0.08 | 100.29 ± 0.19 | — |
| Appearance | Milky/Stable | Milky/Stable | Milky/Stable | Sedimentation | Precipitate |
| | suspension | suspension | suspension | | |

1:5 ratio

| | | | | | |
|---|---|---|---|---|---|
| Z-Average (nm) | 473 ± 3.9 | 630 ± 16.6 | 756 ± 53.2 | — | — |
| PI | 0.047 ± 0.046 | 0.087 ± 0.082 | 0.232 ± 0.198 | — | — |
| ζ -potential (mV) | +80 ± 0.84 | — | — | — | — |
| CBD content (%) | 111.65 ± 0.01 | — | 116.48 ± 0.05 | 101.21 ± 0.15 | — |
| Appearance | Milky/Stable | Milky/Stable | Milky/Stable | Sedimentation | Precipitate |
| | suspension | suspension | suspension | | |

1:10 ratio

| | | | | | |
|---|---|---|---|---|---|
| Z-Average (nm) | — | — | — | — | — |
| PI | — | — | — | — | — |
| ζ -potential (mV) | — | — | — | — | — |
| CBD content (%) | — | — | — | — | — |

| | |
|---|---|
| Appearance | Viscous |

As can be seen from the above results, GCPQ polymers with a mole % palmitoylation (% P) of less than or equal to 31% and typically between 11 and 20% are preferred. Furthermore, GCPQ polymers with a mole % quaternary ammonium groups (% Q) of less than or equal to 17% and typically between 8 and 17% are preferred.

The above results also demonstrate that preferred CBD:GCPQ ratios are above 1:0.5 and ideally between 1:1 and 1:10.

1. A method of treatment comprising administering to a human or animal a composition comprising a cannabinoid and an amphiphilic carbohydrate compound, wherein the composition is intranasally administered to the human or animal body.

2. A composition comprising a cannabinoid and an amphiphilic carbohydrate compound for use in a method of treating a human or animal by intranasal administration.

3. The method or composition for use of claim 1 or 2 wherein the treatment is treatment of a disease of the central nervous system and is preferably epilepsy.

4. The method or composition for use according to any of claims 1 to 3 wherein the treatment is of pain, anxiety or an autoimmune disease.

5. A method or composition for use according to any preceding claim wherein the composition is in the form of nanoparticles.

6. A method or composition for use according to claim 5 wherein the nanoparticles are processed to form nano-in-microparticles.

7. A method or composition for use according to claim 6 wherein the composition is made by a process for forming nano-in-microparticles which is selected from spray-drying, lyophilisation, adding nanoparticles to powders to form granules, preferably wherein the process is spray-drying.

8. A method or composition for use according to claim 6 or 7 wherein the mean size of the nanoparticles in the nano-in-microparticles composition is under 1000 nm, preferably wherein the size of the nanoparticles is under 500 nm.

9. A method or composition for use according to any one of claims 6 to 8 wherein the median volume distribution, D₅₀, of the microparticles in the nano-in-microparticulate composition are between 5 to 30 μm, preferably between 10 to 25 μm.

10. A method or composition for use according to claim 9 wherein less than 10% of particles are below 10 μm.

11. A method or composition for use according to any one of claims 6 to 10 wherein the polydispersity of the nanoparticles within the nano-in-microparticles is less than 0.5, preferably less than 0.2, and more preferably less than 0.1.

12. A method or composition for use according to any one of claims 6 to 11 wherein the nanoparticles within the nano-in-microparticles are a colloidally stable formulation.

13. A method or composition for use according to claim any one of claims 6 to 12 wherein the nano-in-microparticles are in the form of a dry powder.

14. A method or composition for use according to claim 13 wherein the nano-in-microparticles are compatible with nasal spray devices.

15. A method or composition for use according to any one of claims 6 to 14 wherein the nano-in-microparticles have a spherical, hollow or irregular morphology.

16. A method or composition for use according to any preceding claim wherein the ratio of cannabinoid to amphiphilic carbohydrate compound is between 0.5:10 to 5:10 g g⁻¹.

17. A method or composition for use according to claim 16 wherein the ratio of cannabinoid to amphiphilic carbohydrate compound is 1:10 to 3:10 g g⁻¹, preferably wherein the ratio is around 1:5 g g⁻¹.

18. A method or composition for use according to any preceding claim wherein the cannabinoid is delivered to the brain of the human or animal body.

19. A method or composition for use according to any preceding claim wherein the cannabinoid is a non-psychoactive cannabinoid and is preferably cannabidiol.

20. A method or composition for use according to any preceding claim wherein the amphiphilic carbohydrate compound is a chitosan derivative.

21. A method or composition for use according to any preceding claim wherein the amphiphilic carbohydrate compound comprises the general formula: 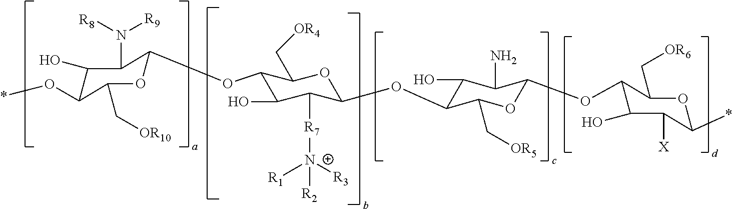
wherein a+b+c+d=1.000 and
a is between 0.01 and 0.970
b is between 0.01 and 0.990,
c is between 0.0001 and 0.970, and
d is between 0.01 and 0.990;
and wherein:
X is a hydrophobic group;
R₁, R₂ and R₃ are independently selected from a substituted or unsubstituted alkyl group;
R₄, R₅, R₆ and R₁₀ are independently selected from hydrogen, a substituted or unsubstituted alkyl group, a substituted or unsubstituted ether group, or a substituted or unsubstituted alkene group;
R₇ may be present or absent and, when present, is an unsubstituted or substituted alkyl group, an unsubstituted or substituted amine group or a substituted or unsubstituted amide group;
R₃ and R₉ are independently selected from hydrogen and either a substituted or unsubstituted alkyl group, a substituted or unsubstituted ether group, or a substituted or unsubstituted alkene group;
or a salt thereof.

22. A method or composition for use according to claim 21 wherein the amphiphilic carbohydrate compound is quaternary ammonium palmitoyl glycol chitosan (GCPQ).

23. A pharmaceutical composition suitable for intranasal administration comprising an amphiphilic carbohydrate compound and a non-psychoactive cannabinoid and one or more pharmaceutically acceptable excipients.

24. A pharmaceutical composition according to claim 23 wherein the cannabinoid is selected from the group consisting of cannabidiol (CBD), cannabigerol (CBG), cannabidiolic acid (CBDA), cannabidivarin (CBDV), cannabichromene (CBC) and combinations thereof.

25. A pharmaceutical composition according to claim 24 wherein the cannabinoid is cannabidiol (CBD).

26. A pharmaceutical composition according to any of claims 23 to 25 wherein the excipient is selected from the group consisting of tonicity enhancers, preservatives, solubilisers, non-toxic excipients, demulcents, sequestering agents, pH adjusting agents, co-solvents, viscosity building agents and combinations thereof.

27. A pharmaceutical composition according to any of claims 23 to 26 wherein the amphiphilic carbohydrate compound is GCPQ.

28. A pharmaceutical composition according to any of claims 23 to 27 wherein the composition is in the form of nanoparticles.

29. A pharmaceutical composition according to claim 28 wherein the nanoparticles are processed to form nano-in-microparticles.

30. A pharmaceutical composition according to claim 28 or 29 wherein the mean size of the nanoparticles in the nano-in-microparticles composition are under 1000 nm, preferably wherein the size of the nanoparticles is under 500 nm.

31. A pharmaceutical composition according to any one of claims 28 to 30 wherein the median volume distribution, D₅₀, of the microparticles in the nano-in-microparticulate composition are between 5 to 30 μm, preferably between 10 to 25 μm.

32. A pharmaceutical composition according to claim 31 wherein less than 10% of particles are below 10 μm.

33. A pharmaceutical composition according to any one of claims 29 to 32 wherein the polydispersity of the nanoparticles within the nano-in-microparticles is less than 0.5, preferably less than 0.2, and more preferably less than 0.1.

34. A pharmaceutical composition according to any one of claims 29 to 33 wherein the nanoparticles within the nano-in-microparticles are a colloidally stable formulation.

35. A pharmaceutical composition according to claim any one of claims 29 to 33 wherein the nano-in-microparticles are in the form of a dry powder.

36. A pharmaceutical composition according to claim 35 wherein the nano-in-microparticles are compatible with nasal spray devices.

37. A pharmaceutical composition according to any one of claims 29 to 36 wherein the nano-in-microparticles have a spherical morphology.

38. A pharmaceutical composition according to any one of claims 23-37 wherein the ratio of cannabinoid to amphiphilic carbohydrate compound is between 0.5:10 to 5:10 g g⁻¹.

39. A pharmaceutical composition according to claim 38 wherein the ratio of cannabinoid to amphiphilic carbohydrate compound is 1:10 to 3:10 g g⁻¹, preferably wherein the ratio is around 1:5 g g⁻¹.

40. A pharmaceutical composition according to any of claims 23 to 39 wherein the cannabidiol is stable for at least 4 weeks at 4° C.

41. A pharmaceutical composition according to any of claims 23 to 40 wherein the cannabidiol is stable for at least 4 weeks at 25° C.

42. A pharmaceutical composition according to any of claims 23 to 41 wherein the cannabidiol is least 99% recovered after being stored 4 weeks at 4° C.

43. A pharmaceutical composition comprising GCPQ and a cannabinoid and one or more pharmaceutically acceptable excipients.

44. A pharmaceutical composition according to claim 43 wherein the GCPQ has a palmitoylation level in the range 11-31 mole %.

45. A pharmaceutical composition according to claim 43 or 44 wherein the GCPQ has a quaternisation level in the range 8-17 mole %.

46. A pharmaceutical composition according to any of claims 43 to 45 wherein the ratio of GCPQ:cannabinoid is between 1:1 and 1:10.

47. A pharmaceutical composition according to claim 46 wherein the cannabinoid is selected from the group consisting of cannabidiol (CBD), cannabigerol (CBG), cannabidiolic acid (CBDA), cannabidivarin (CBDV), cannabichromene (CBC) and combinations thereof.

48. A pharmaceutical composition according to claim 47 wherein the cannabinoid is cannabidiol (CBD).

49. A composition comprising GCPQ and a cannabinoid wherein the GCPQ has a palmitoylation level in the range 11-31 mole % and a quaternisation level in the range 8-17 mole %, and the ratio of GCPQ:cannabinoid is between 1:1 and 1:10.
